(19) United States
(12) Patent Application Publication
(10) Pub. No.: US 2015/0246170 A1
(43) Pub. Date: Sep. 3, 2015

(54) TARGET-DIRECTED, MAGNETICALLY ENHANCED SYSTEM FOR DETOXIFICATION OF PATIENTS

(71) Applicants: Wenliang Miao, Zhejiang (CN); Nan Wang, Zhejiang (CN); Yinan Miao, Zhejiang (CN); Wei Li, Zhejiang (CN)

(72) Inventors: Wenliang Miao, Zhejiang (CN); Nan Wang, Zhejiang (CN); Yinan Miao, Zhejiang (CN); Wei Li, Zhejiang (CN)

(73) Assignee: HANGZHOU EVERLONG BIOTECHNICS, CO., LTD., Hangzhou, Zhejiang (CN)

(21) Appl. No.: 13/977,249

(22) PCT Filed: Jun. 14, 2011
(86) PCT No.: PCT/CN2011/075748

§ 371 Date: Sep. 10, 2013

(51) Int. Cl. A61M 1/36

(52) U.S. Cl. CPC A61M 1/3618; A61M 1/3679

(57) ABSTRACT

A target-directed, magnetically enhanced system and method for detoxification of patients. The system comprises a first fluid circuit for circulation of biological fluid and a second fluid circuit for co-circulation of biological fluid. The first fluid circuit comprises, in the following order: a first fluid circuit inlet (10), a reaction chamber (2), an equipment comprising one or more elements that separate the magnetic microspheres from the biological fluid, and a first fluid circuit outlet (13). The second fluid circuit initiates after the first fluid circuit inlet (10) and terminates before the first fluid circuit outlet (13). The system and method can be used to quickly and effectively remove toxins, infectious agents, allergens, cancer cells, and other unwanted substances from a patient, and provide extracorporeal blood or plasma treatment.

### FIELD OF INVENTION

The present invention relates generally to systems and methods for detoxification of patients.

### BACKGROUND

The capability to rapidly and effectively remove toxic substances from patients can be life-saving. In many instances, even trace levels of poisons, such as snake toxin, can be lethal. In addition, the capability to effectively eliminate tumor cells, particularly metastatic cells, bears great therapeutic potential. Therefore, new generations of systems and methods capable of depleting toxins and cancerous cells from human bodies in a specific, rapid and efficient manner are in huge demand.

### BRIEF SUMMARY

The present invention provides systems and methods for removing toxic substances from patients via extracorporeal circulation of biological fluid. In one aspect, the present invention provides a target-directed, magnetically enhanced system that can quickly and effectively remove toxins, infectious agents, allergens, cancer cells, and other unwanted substances from patients. Also provided are components of the system related to this invention.

In a preferred embodiment, the target-directed, magnetically enhanced system provides in vitro treatment for patients utilizing circulating blood and/or plasma.

In one embodiment, the target-directed, magnetically enhanced system comprises:

a reaction chamber comprising:

a first fluid circuit inlet for receiving biological fluid from a subject,

a first fluid circuit outlet for returning the biological fluid back to the subject,

a second fluid circuit outlet allowing the biological fluid to flow out of the reaction chamber and enter into a second fluid circuit, and

a second fluid circuit inlet for returning the biological fluid from the second fluid circuit to the reaction chamber;

a reservoir of magnetic microspheres that specifically capture target molecules to be removed from the biological fluid; and

equipment comprising one or more elements that separate the magnetic microspheres from the biological fluid, whereby the equipment allows the flow-through of the biological fluid but inhibits the passage of the magnetic microspheres, and thereby prevents the magnetic microspheres from entering into the subject;

wherein the system comprises a first fluid circuit for circulation of the biological fluid, and the first fluid circuit comprises, in the following order: the first fluid circuit inlet, the reaction chamber, said equipment, and the first fluid circuit outlet; and

wherein the system comprises a second fluid circuit for co-circulation of the biological fluid and the microspheres into, through, and out of the reaction chamber, wherein the second fluid circuit initiates after the first fluid circuit inlet and terminates before the first fluid outlet, wherein the reservoir is positioned along the second fluid circuit.

In a preferred embodiment, the surfaces of the magnetic microspheres are conjugated with antibodies that bind specifically to target molecules in the biological fluids.

In one specific embodiment, a plurality of the systems of the invention can be connected in series.

Another aspect of the invention provides a method for removing target molecules from a subject via extracorporeal circulation of biological fluid of the subject. In one embodiment, the method comprises:

a) receiving biological fluid from a subject, wherein the biological fluid comprises target molecules to be removed;

b) providing magnetic microspheres that bind specifically to the target molecules to be removed from the biological fluid;

c) directing the biological fluid and the magnetic microspheres to a system comprising:

a reaction chamber, comprising:

a first fluid circuit inlet for receiving biological fluid from a subject,

a first fluid circuit outlet for recirculating the biological fluid back to the subject,

a second fluid circuit outlet allowing the biological fluid to flow out of the reaction chamber and enter into a second fluid circuit, and

a second fluid circuit inlet for returning the biological fluid from the second fluid circuit to the reaction chamber;

a reservoir of magnetic microspheres that bind specifically to the target molecules of the biological fluid; and

equipment comprising one or more elements that separate the magnetic microspheres from the biological fluid, whereby the equipment allows the flow-through of the biological fluid but inhibits the passage of the magnetic microspheres, and thereby prevents the magnetic microspheres from entering into the subject;

wherein the system comprises a first fluid circuit for circulation of the biological fluid, and the first fluid circuit comprises, in the following order: the first fluid circuit inlet, the reaction chamber, said equipment, and the first fluid circuit outlet; and

wherein the system comprises a second fluid circuit for co-circulation of the biological fluid and the microspheres into, through, and out of the reaction chamber, wherein the second fluid circuit initiates after the first fluid circuit inlet and terminates before the first fluid outlet, wherein the reservoir is positioned along the second fluid circuit; and

d) returning the biological fluid back to the subject.

### BRIEF DESCRIPTION OF THE DRAWINGS

FIG. 1 schematically illustrates one embodiment of the target-directed, magnetically enhanced system of the present invention.

FIG. 2 shows one embodiment of the reaction chamber of the present invention.

FIG. 3 shows one embodiment of the filter element of the present invention.

FIG. 4 shows one embodiment of the filter element of the present invention.

FIG. 5 shows a cross-sectional view of one embodiment of the target-directed, magnetically enhanced system of the present invention.

FIG. 6 shows one embodiment of the filter element, comprising a plurality of filtering tubes.

FIG. 7 shows one embodiment of the filtering tube of the present invention.

FIG. 8 shows one embodiment of the reaction chamber of the present invention.

FIG. 9 shows one embodiment of the reaction chamber of the present invention.

FIG. 10 shows one embodiment of the reaction chamber of the present invention.

FIG. 11 schematically illustrates one embodiment of the target-specific, magnetically enhanced system of the present invention.

FIG. 12 shows one embodiment of the device for separating biological fluid from magnetic microspheres.

FIG. 13 shows one embodiment of the device for separating biological fluid from magnetic microspheres.

FIG. 14 shows one embodiment of the device for separating biological fluid from magnetic microspheres.

FIG. 15 shows one embodiment of the reaction chamber, which employs an external magnet.

### DETAILED DESCRIPTION

The present invention provides systems and methods for detoxification of patients via extracorporeal circulation of biological fluid. In one aspect, the present invention provides a target-specific, magnetically enhanced system that can quickly and effectively remove toxins, infectious agents, allergens, cancer cells, and other unwanted substances from a patient in a target-specific manner. Also provided are components of the system of the present invention. In a preferred embodiment, the target-directed, magnetically enhanced system provides extracorporeal blood or plasma treatment. Another aspect of the invention provides a method for removing target molecules from a subject via extracorporeal circulation of biological fluid of the subject.

Advantageously, the present invention can rapidly and effectively remove toxic substances and cancer cells from a patient in a safe and target-specific manner. The present invention is particularly useful for the treatment of hematological cancer and/or lymphoproliferative disorder.

### Target-Directed, Magnetically Enhanced System for Removing Toxic Substances

One aspect of the present invention provides a target-specific, magnetically enhanced system that can quickly and effectively remove toxins, infectious agents (including viruses, bacteria, fungus and other microorganisms), allergens, cancer cells, and other unwanted substances from a patient. In a preferred embodiment, the target-specific, magnetically enhanced system provides extracorporeal blood or plasma treatment.

In one embodiment, the target-specific, magnetically enhanced system comprises:

a reaction chamber, comprising:

a first fluid circuit inlet for receiving biological fluid from a subject,

a first fluid circuit outlet for returning the biological fluid back to the subject,

a second fluid circuit outlet allowing the biological fluid to flow out of the reaction chamber and enter into a second fluid circuit, and

a second fluid circuit inlet for returning the biological fluid from the second fluid circuit to the reaction chamber;

a reservoir of magnetic microspheres that specifically capture target molecules to be removed from the biological fluid; and

equipment comprising one or more elements that separate the magnetic microspheres from the biological fluid, whereby the equipment allows the flow-through of the biological fluid but inhibits the passage of magnetic microspheres, and thereby prevents the magnetic microspheres from entering into the subject;

wherein the system comprises a first fluid circuit for circulation of the biological fluid, and the first fluid circuit comprises, in the following order: the first fluid circuit inlet, the reaction chamber, said equipment, and the first fluid circuit outlet; and

wherein the system comprises a second fluid circuit for co-circulation of the biological fluid and the microspheres into, through, and out of the reaction chamber, wherein the second fluid circuit initiates after the first fluid circuit inlet and terminates before the first fluid outlet, wherein the reservoir is positioned along the second fluid circuit.

In a preferred embodiment, the biological fluid is blood (including whole blood, plasma, and serum).

In one specific embodiment, a plurality of the systems of the invention can be connected in series.

In one embodiment, the second fluid circuit terminates before the equipment that separates the magnetic microspheres from the biological fluid. In one embodiment, the equipment can be, for example, a size-based filter and a magnetic-based device capable of capturing magnetic microspheres.

In one embodiment, the system further comprises a magnetic-based device capable of capturing magnetic microspheres, wherein the magnetic-based device is positioned along the second fluid circuit. Preferably, a valve is coupled to the magnetic-based device. Preferably, the valve can be opened or closed in a controlled manner to remove used magnetic microspheres bound to target molecules.

Used, target-bound magnetic microspheres removed from the second fluid circuit can be disposed. In an alternative embodiment, used magnetic microspheres can be recycled. In one embodiment, the magnetic-based device is connected to a recycling device capable of removing the bound target molecules from the magnetic microspheres, wherein the recycling device is positioned to receive the captured target-bound magnetic microspheres continuously, or in a controlled manner. In one embodiment, the recycling device contains a very high concentration of molecules that can bind to target molecules. In one embodiment, the recycling device is positioned along the second fluid circuit so that recycled magnetic microspheres can be fed back into the second fluid circuit.

In certain embodiments, the system further comprises one or more of valves including, but not limited to,

a) a valve coupled to the first fluid circuit inlet, wherein the valve is positioned to prevent the flow of the biological fluid and/or the magnetic microspheres to the subject;

b) a valve coupled to the first fluid circuit outlet;

c) a valve coupled to the second fluid circuit outlet;

d) a valve coupled to the second fluid circuit inlet;

e) a valve coupled to the equipment that separates magnetic microspheres from the biological fluid; and

f) a valve coupled to the reservoir.

Preferably, the valve(s) can be manipulated to obtain a desired switch on/off time.

In one embodiment, the system further comprises means for facilitating the mixing and therefore the interaction of biological fluid (such as blood) and magnetic microspheres. In one embodiment, a magnetic field is provided to stir magnetic microspheres in a desired manner. The magnetic field can be generated by one or more magnets located inside and/or outside of the reaction chamber. In one embodiment, the reaction chamber comprises a stirring element so that the magnetic microspheres and/or biological fluid are stirred continuously or periodically. The stirrer can be in any suitable shape (such as a bar, beads) and made of any suitable material (such as metal, plastic). The working condition of the stirring procedure can be purpose-designed featuring multi-dimensional, rate, and duration as long as sufficient mixing can be achieved.

In one embodiment, the system further comprises a cleaning device and/or a waste fluid collector.

In one embodiment, the present invention does not encompass the immunological-based blood treatment device disclosed in Chinese Utility Mode Patent No. ZL 2005 2 0040240.0.

Equipment for Separating Magnetic Microspheres from Biological Fluid

The system of the present invention comprises equipment that can effectively separate magnetic microspheres from biological fluids (such as blood). The equipment allows the flow-through of biological fluids or components therein (such as blood cells), but blocks the passage of the magnetic microspheres.

In one embodiment, a filter element is provided for separating magnetic microspheres from the biological fluid. The filter element can be made of any suitable materials. In one embodiment, the filter element is made of a semi-permeable membrane.

In one embodiment, the size of the pore can be of any size that is larger than the non-targeted components of the biological fluid to be returned to the subject, but is smaller than the magnetic microspheres. In one embodiment, the size of the pore is larger than about 7, 8, 9, 10, 13, 15, 17, 20, 25, 30, 50, or 60 μm (such as in terms of diameter). In one embodiment, the size of the pore is smaller than about 9, 10, 11, 12, 14, 16, 18, 20, 30, 40, 60, or 80 μm (such as in terms of diameter). In one embodiment, the size of the pore is about 10 to about 80, about 10 to about 70, about 10 to about 50, about 10 to about 40, about 8 to about 30, about 10 to about 20, about 10 to about 15, about 15 to about 30, or about 20 to about 30 μm (such as in terms of diameter).

In one embodiment, the filter element is positioned in a manner that prevents magnetic microspheres from entering into the subject. In one embodiment, the filter element is located inside the reaction chamber and is positioned in a manner that completely separates the reaction chamber into separate compartments. As a result, magnetic microspheres are confined in a closed fluid circuit not in contact with the subject, whereas biological fluid (such as blood) can pass freely through the filter element for returning back to the subject.

Exemplified embodiments of the filter element of the present invention are illustrated in FIGS. 3 and 4.

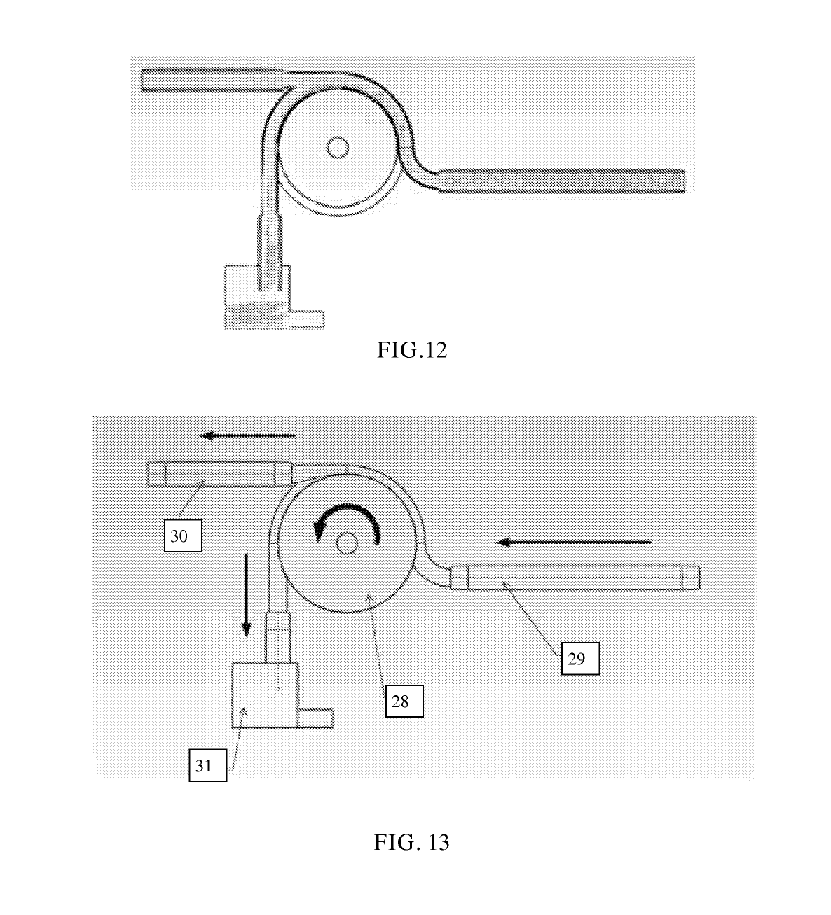

In one embodiment, a magnetic-based device is provided for separating magnetic microspheres from the biological fluid. The magnetic-based device can capture magnetic microspheres, but does not capture or adsorb biological fluid or components therein (such as blood cells). As a mixture of biological fluid and magnetic microspheres passes through the magnetic-based device, the magnetic microspheres are captured into the magnetic-based device, and, thereby removed from the biological fluid. The magnetic-based device can be located inside or outside of the reaction chamber. Exemplified embodiments of the magnet-based separation device are illustrated in FIGS. 12 and 13.

In one embodiment, the magnet-based device is provided to remove used magnetic microspheres. In one specific embodiment, the magnetic-based device is positioned along the second fluid circuit in which the biological fluid co-circulates with magnetic microspheres.

### Cycler

In one embodiment, a fresh source of magnetic microspheres can be pumped into and circulated along the fluid circuit, and subsequently drained from the fluid circuit after each treatment cycle with the use of a cycler. In one embodiment, the cycler is a pump. In one embodiment, a cycler is coupled to, or along with, a fluid path or paths in any suitable manner such that fluid flow can be automatically controlled. In another embodiment, the cycler can determine the volume of fluid delivered.

In one embodiment, one or more pumps and valves can be coupled to the treatment system to provide efficient and effective automatic control of flowing of biological fluid and/or magnetic microspheres. In one embodiment, a valve is coupled to the first fluid circuit inlet for receiving biological fluid from the subject, and the valve is configured to prevent the backflow of fluid and magnetic microspheres to the subject. In one embodiment, a valve is coupled to the reservoir of magnetic microspheres so that the magnetic microspheres can be released at desired time points and/or at a desired flow rate. In one embodiment, a valve is coupled to a magnetic-based device (such as a magnet) that captures used, target-bound microspheres, and the valve can be switched on periodically to remove used magnetic microspheres at desired time points. The flow of magnetic microspheres and biological fluid can be automatically controlled or can be controlled by the end user.

### Monitor and Sensor

In one embodiment, the system further comprises one or more sensors. In one embodiment, the sensor is capable of detecting the presence of magnetic microspheres in the biological fluid. In one embodiment, a sensor is located downstream of the device that separates magnetic microspheres from the biological fluid. If it is detected that the returning biological fluid contains magnetic microspheres, the biological fluid will not be forwarded to the subject, but will be sent back to a device that captures magnetic microspheres. In one embodiment, a sensor is provided to detect signals, such as, the volume, pressure, pH and/or flow rate of the magnetic microspheres and/or the biological fluid. In one embodiment, signals detected by the sensor are sent to a valve or a cycler so that the entry, release and/or flow rate of the fluid (such as blood, therapeutic solution, and mixtures thereof) can be controlled in a desirable manner.

### Dialysis Equipment

In one embodiment, the system further comprises equipment (such as a dialyzer) that adjusts water and electrolyte content and removes unwanted small molecular substances from the subject. In one embodiment, the reaction chamber is coupled to dialysis equipment. In one embodiment, dialysis solution or other therapeutic composition can be fed into the reaction chamber, via an input module that is the same or different from the input module for magnetic microspheres. In a further embodiment, buffers such as phosphate and bicarbonate, can be added.

The dialysate solution useful according to the present invention can include osmotic agents, such as dextrose, and/or electrolytes including, but not limited to, calcium, sodium, and potassium.

In one embodiment, the system further comprises adsorption or binder materials that can effectively remove unwanted substances such as carbon, urea, and ammonia from the biological fluid. The adsorption/binder materials are known in the art. For instance, materials that can bind to urea include, but are not limited to, alkenylaromatic polymers containing phenylglyoxal and polymeric materials containing tricarbonyl functionality.

### Target-Specific, Magnetic Microspheres

In one embodiment, the present invention provides magnetic microspheres that bind specifically to, and thereby capture, target molecules or cells displaying certain types of surface antigens. Target molecules can be any unwanted substances including, but not limited to, protein, peptides, ligands, antibodies, antigens, glycoproteins, hormones, toxins, compounds, nucleic acid molecules (including ssDNA, dsDNA, and RNA), carbohydrates, lipids, and infectious agents.

In one embodiment, the magnetic microspheres of the present invention are surface-coated with molecules (e.g., antigens, antibodies, receptors, ligands, nucleic acid molecules) that bind specifically to the target. In one embodiment, the surface of the magnetic microspheres is further covalently conjugated with an —OH, —COOH, and/or —NH group to facilitate and stabilize the interaction with target molecules of protein origins. In one embodiment, the magnetic microspheres are also coated with an additional therapeutic agent.

The magnetic microsphere can be of any size suitable for practicing the invention. In one embodiment, the magnetic microsphere is larger than the particle size of the biological fluid or cellular and acellular components thereof (such as blood cells). In one embodiment, the magnetic microsphere has a size ranging from about 0.2 to 100 μm. In one embodiment, the magnetic microsphere has a size larger than about 10, 12, 15, 17, 20, 22, 25, 30, 35, 40, 50, 60, or 70 μm (such as in terms of diameter). In one embodiment, the magnetic microsphere has a size smaller than about 13, 15, 17, 20, 25, 30, 40, 50, 60, 70, 80, 90, or 100 μm (such as in terms of diameter). In one embodiment, the magnetic microsphere has a size of about 10 to about 100, about 10 to about 90, about 10 to about 70, about 10 to about 50, about 10 to about 30, about 10 to about 20, about 15 to about 30, about 10 to about 15, about 15 to about 20, or about 20 to about 30 μm (such as in terms of diameter).

In one embodiment, the magnetic particles can be made of elements including, but not limited to, earth elements such as neodymium and samarium, compounds such as neodymium-iron-boron and samarium-cobalt, and ferromagnetic materials such as iron, permalloy, superpermalloy, cobalt, nickel, steel, and alnico. In one embodiment, the magnetic microspheres useful according to the present invention include any particles that can be caused to move under the influence of a magnetic field.

In one embodiment, the magnetic microspheres do not bind specifically to healthy, non-targeted blood cells including, but not limited to, healthy red blood cells, B lymphocytes, T lymphocytes, and platelets, and cellular and acellular components therein. In one embodiment, the magnetic microspheres do not bind specifically to healthy, non-targeted cells including, but not limited to, healthy granulocytes, erythrocytes, thrombocytes, macrophages, mast cells, lymphocytes.

In one embodiment, the target molecule is a toxic substance (or an epitope therein) including, but not limited to, animal, plant and/or synthetic toxins including, but not limited to, snake toxins, spider toxins, scorpion toxins, and mushroom toxins.

In one embodiment, the target molecule is an epitope displayed on the surface of cancer cells including, but not limited to, breast, lung, colon, gastric, esophagus, bone marrow, stomach and liver carcinoma cells. In one specific embodiment, the target molecule is an epitope displayed on the surface of hematologic tumor cells and/or cancer cells of lymphoproliferative disorder including, but not limited to, leukemia, lymphoma, lymphocytic leukemia, acute and chronic myelogenous leukemia, myelodysplastic syndrome, myeloproliferative disease, multiple myeloma, non-Hodgkin's lymphoma, Burkitt's lymphoma, and follicular lymphoma cells. In one specific embodiment, the target molecule can be an epitope displayed on the surface of metastatic cancer cells. In one specific embodiment, the target molecule is an epitope displayed on the surface of cancer stem cells.

In one embodiment, the target molecule can be hormones such as growth factors (such as vascular endothelial growth factor (VEGF)), kinases, cytokins, and pro-inflammatory agents.

In one embodiment, the target molecule is an epitope displayed on a viral envelop, and/or a nucleic acid molecule of viruses including, but not limited to, respiratory syncytial virus, rhinovirus, HIV virus, hepatitis viruses, oncoviruses, human T-lymphotropic virus Type I (HTLV-1), bovine leukemia virus (BLV), Epstein-Barr virus, herpes simplex virus 1, herpes simplex virus 2, coronavirus, and poliovirus.

In one embodiment, the target molecule is a bacterial antigen, and/or a bacterial nucleic acid molecule including, but not limited to, those found in such bacteria species including, Chlamydia trachomatis, Chlamydia pneumonaie, M. tuberculosis, and H. pylori.

In one embodiment, the magnetic microsphere is attached with an antibody, an antibody fragment, or a fusion protein thereof that binds specifically to the target antigens (such as a protein, peptide).

Antibodies applicable according to the present invention can be in various forms, including a whole immunoglobulin, an antibody fragment such as Fab, Fab′, F(ab′)₂, Fv region containing fragments, and similar fragments, as well as a single chain antibody that includes the variable domain complementarity determining regions (CDR), and similar forms. Antibodies within the scope of the invention can be of any isotype, including IgG, IgA, IgE, IgD, and IgM. IgG isotype antibodies can be further subdivided into IgG1, IgG2, IgG3, and IgG4 subtypes. IgA antibodies can be further subdivided into IgA1 and IgA2 subtypes.

In one embodiment, the magnetic microsphere of the present invention is coated with nucleic acid molecules that hybridize, under stringent conditions, with a target nucleic acid molecule. In another embodiment, the magnetic microsphere is coated with aptamers specific for a target molecule.

In one embodiment, the magnetic microsphere of the present invention is coated with nucleic acid molecules complementary to the full length, or a fragment of, the target nucleic acid molecule. In one embodiment, the magnetic microsphere of the present invention is coated with nucleic acid molecules that complementary to at least 10, 20, 30, 40, 50, 60, 70, 80, 90, 100, 200, 300, 400, 500, 600, 700, 800, 900, 1000, 1500, 2000, 2500, or 3000 contiguous nucleic acids of a target nucleic acid molecule.

As used herein, “stringent” conditions for hybridization refers to conditions whereby hybridization is typically carried out overnight at 20-25 C below the melting temperature (Tm) of the DNA hybrid in 6×SSPE, 5×Denhardt's solution, 0.1% SDS, 0.1 mg/ml denatured DNA. The melting temperature, Tm, is described by the following formula (Beltz et al., 1983):

Tm=81.5 C+16.6 Log [Na+]+0.41(% G+C)−0.61(% formamide)−600/length of duplex in base pairs.

Washes are typically carried out as follows:

(1) Twice at room temperature for 15 minutes in 1×SSPE, 0.1% SDS (low stringency wash).

(2) Once at Tm-20 C for 15 minutes in 0.2×SSPE, 0.1% SDS (moderate stringency wash).

In one embodiment, the magnetic microsphere of the present invention is coated with receptor molecules that bind to target ligands. In another embodiment, the magnetic microsphere of the present invention is coated with ligands that bind to target receptors.

“Specific binding” or “specificity” refers to the ability of an antibody or other agent to exclusively bind to an epitope presented on an antigen while having relatively little non-specific affinity with other proteins or peptides. Specificity can be relatively determined by binding or competitive binding assays, using, e.g., Biacore instruments. Specificity can be mathematically calculated by, e.g., an about 10:1, about 20:1, about 50:1, about 100:1, 10.000:1 or greater ratio of affinity/avidity in binding to the specific antigen versus nonspecific binding to other irrelevant molecules.

### Methods for Removing Toxic Substances Via Extracorporeal Circulation of Biological Fluid

Another aspect of the invention provides a method for removing target molecules from a subject via extracorporeal circulation of biological fluid. The extracorporeal circulation of biological fluid is provided using the target-directed, magnetically enhanced system of the present invention. In one embodiment, the method comprises:

a) receiving biological fluid from a subject, wherein the biological fluid comprises target molecules to be removed;

b) providing magnetic microspheres that bind specifically to the target molecules to be removed from the biological fluid;

c) directing the biological fluid and the magnetic microspheres to a system comprising:

a reaction chamber, comprising:

a first fluid circuit inlet for receiving biological fluid from a subject,

a first fluid circuit outlet for returning the biological fluid back to the subject,

a second fluid circuit outlet allowing the biological fluid to flow out of the reaction chamber and enter into a second fluid circuit, and

a second fluid circuit inlet for returning the biological fluid from the second fluid circuit to the reaction chamber;

a reservoir of magnetic microspheres that bind specifically to the target molecules to be removed from the biological fluid; and

equipment comprising one or more elements that separate the magnetic microspheres from the biological fluid, wherein the equipment allows the flow-through of the biological fluid but prevents the passage of the magnetic microspheres, and thereby prevents the magnetic microspheres from entering into the subject;

wherein the system comprises a first fluid circuit for circulation of the biological fluid, and the first fluid circuit comprises, in the following order: the first fluid circuit inlet, the reaction chamber, said equipment, and the first fluid circuit outlet; and

wherein the system comprises a second fluid circuit for co-circulation of the biological fluid and the microspheres into, through, and out of the reaction chamber, wherein the second fluid circuit initiates after the first fluid circuit inlet and terminates before the first fluid outlet, wherein the reservoir is positioned along the second fluid circuit; and

d) returning the biological fluid back to the subject.

In one embodiment, the present invention provides a method for removing target molecules from a subject using a plurality of the systems of the invention, wherein said plurality of the systems are connected in series. In one specific embodiment, step (c) of the method comprises: directing the biological fluid of the subject to a plurality of the systems, wherein the plurality of the treatment systems are connected in series.

The term “subject,” as used herein, describes an organism, including mammals such as primates. Mammalian species that can benefit from the subject methods include, but are not limited to, apes, chimpanzees, orangutans, humans, monkeys; and domesticated and/or laboratory animals such as dogs, cats, horses, cattle, pigs, sheep, goats, chickens, mice, rats, guinea pigs, and hamsters. Typically, the subject is a human.

In one embodiment, the biological fluid includes, but is not limited to, blood, lymph, serum, plasma. In a preferred embodiment, the biological fluid is blood (including, whole blood, serum, and plasma).

### Compositions

The present invention contemplates compositions comprising the magnetic microspheres and, optionally, additionally agents useful for practicing the present invention. In one embodiment, the composition comprises a physiologically tolerable carrier.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. Examples of suitable pharmaceutical carriers are described in “Remington's Pharmaceutical Sciences” by E. W. Martin. In addition, if desired, the composition can contain minor amounts of auxiliary substances such as wetting or emulsifying agents, pH buffering agents and the like which enhance the working efficiency of the active ingredient.

While in certain examples, the systems and methods of the present invention are used to provide extracorporeal blood treatment, it would be readily understood that the therapeutic benefits of the present invention extend to the treatment of other biological fluids.

### EXAMPLES

Following are examples that illustrate procedures for practicing the invention. These examples should not be construed as limiting.

### Example 1

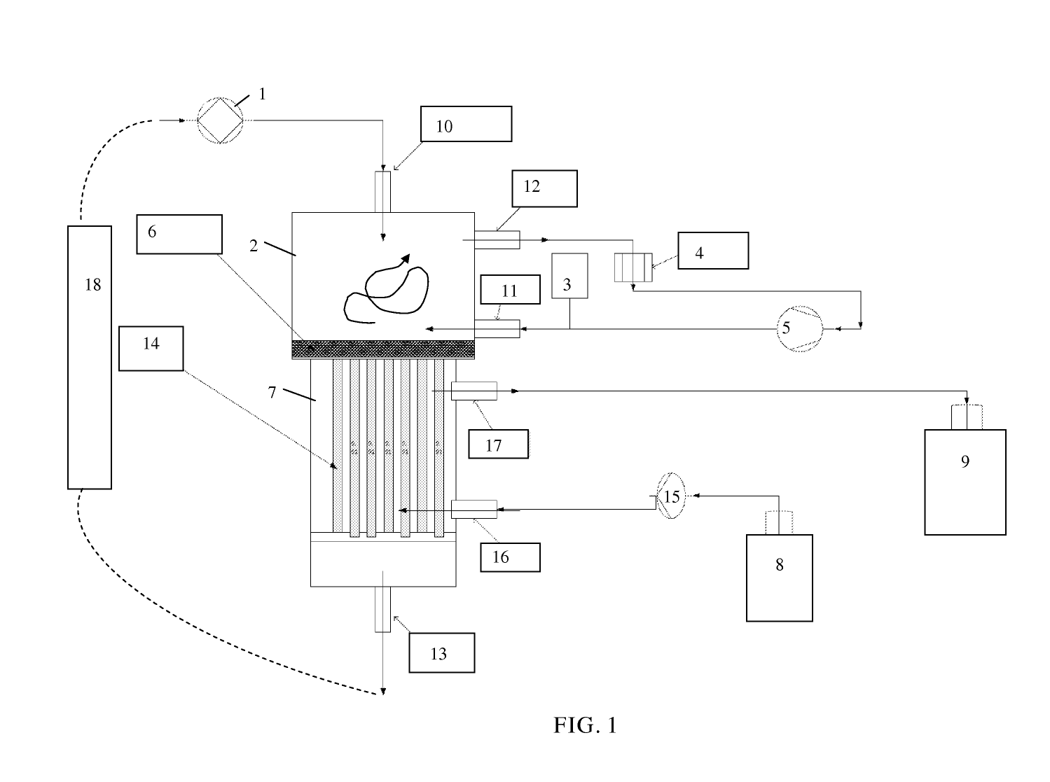

FIG. 1 schematically illustrates one embodiment of the target-directed, magnetically enhanced system of the present invention. The system comprises: a pump (1) for pumping blood, a reaction chamber (2), a reservoir (3) for supplying non-reacted, magnetic microspheres, a magnet (4), a pump (5) for pumping magnetic microspheres, a filter element (6), dialysis equipment (7), a device-cleaning component (8), and a waste fluid collector (9). Various components of the system can be connected by catheters or by any means that allow the passage of blood and/or magnetic microspheres.

The reaction chamber receives untreated blood from the patient (18) via the first fluid circuit inlet (10). The treated blood eventually returns to the patient via the first fluid circuit outlet (13). A pump (1) is provided upstream of the reaction chamber (2) to facilitate the inflow of patient blood. In one embodiment, a valve is coupled to the inlet (10) to prevent fluid (including blood) and magnetic microspheres from exiting the reaction chamber through the inlet (10). The reaction chamber also comprises a second fluid circuit inlet (11) for receiving fresh, non-reacted magnetic microspheres, and a second fluid circuit outlet (12) allowing magnetic microspheres to flow out of the reaction chamber. As shown in FIG. 1, patient blood is fed into the reaction chamber in a direction opposite to which magnetic microspheres are fed into the reaction chamber. This counter-current flow facilitates the interaction between the blood cells and the magnetic microspheres.

As illustrated in FIG. 1, a closed second fluid circuit is provided for the circulation of magnetic microspheres into (via inlet (11)), through, and out of (via outlet (12)) the reaction chamber. The second fluid circuit not only allows continuous circulation of magnetic microspheres and blood, but also allows rapid and effective removal of spent magnetic microspheres. A pump (5) is provided along the second fluid circuit to facilitate the unidirectional flow of magnetic microspheres. A reservoir (3), which supplies a fresh source of non-reacted magnetic microspheres, is located upstream of the reaction chamber. Preferably, the reservoir is coupled to a valve so that magnetic microspheres can be released into the second fluid circuit at desired time points and in a controlled manner. Before patient blood is fed into the reaction chamber, magnetic microspheres can self-circulate along the second fluid path continuously. After patient blood is fed into the reaction chamber, blood cells can also circulate, with magnetic microspheres, along the second fluid path; the co-circulation of blood cells and magnetic microspheres allows sufficient interaction between the blood cells and magnetic microspheres. In one embodiment, a magnet (4) is positioned along the second fluid circuit. The magnet captures magnetic microspheres, but does not attract patient blood cells. Preferably, a valve is coupled to the magnet. The valve can be opened periodically so that spent magnetic microspheres can be removed from the second fluid circuit; meanwhile, fresh, non-reacted magnetic microspheres can be added into the circuit from the reservoir.

A filter element (6) is provided to prevent magnetic microspheres from entering into the patient. The filter element allows the passage of patient blood cells, but completely blocks the passage of magnetic microspheres. In one embodiment as illustrated in FIG. 1, the filter element is positioned in a sealed manner against the inner walls of the reaction chamber, and separates the reaction chamber into two compartments. In this way, the magnetic microspheres are confined in the upper compartment, and cannot enter into the lower compartment. Blood cells can pass through the filter element and enter freely into the fluid circuit to react with magnetic microspheres.

The filter element can be of a variety of shapes and can be positioned in a variety of manners. FIG. 3 shows one embodiment of the filter element, which is in a ring structure, and can be positioned in a manner that separates the reaction chamber into compartments. FIG. 4 shows another embodiment of the filter element, which is a coiled, hollow tube. One lumen of the coiled tube is connected to the second fluid circuit inlet (11), and the other lumen is connected to the second fluid circuit outlet (12). In this way, a closed second fluid circuit for the co-circulation of magnetic microspheres is formed. The magnetic microspheres are confined inside the second fluid path formed by the filter element, the magnet, the pump, the reservoir, and the catheters connecting the above-mentioned components.

In a preferred embodiment as illustrated in FIG. 1, dialysis equipment (7) is coupled to the lower compartment of the reaction chamber. The dialysis equipment contains therein a plurality of capillary tubes (14) made of semi-permeable membranes. In one embodiment, the semi-permeable membranes can remove unwanted substances (e.g., toxins, contaminants, lipids, and toxic products of metabolism such as urea, creatinine, ammonia, and uric acid) from blood via diffusion, convection, and/or absorption. In one embodiment, an effective amount of dialysate can be added into the dialysis equipment to remove excess water from the blood, and/or to adjust the concentration and/or amount of electrolytes including, but not limited to, calcium, sodium, and potassium. The water, lipid, and electrolyte content can also be adjusted via ultrafiltration and/or osmotic pressure.

In one embodiment, the dialysis equipment (7) is connected to a waste fluid collector (9). In this way, waste fluid can be removed from the blood treatment device via the outlet (17). The dialysis equipment is also connected to a cleaning component (8). A pump (15) is provided to pump cleaning composition into the dialysis equipment via the inlet (16). The dialysis equipment further comprises a first fluid circuit outlet (13) that returns the treated blood back to the patient.

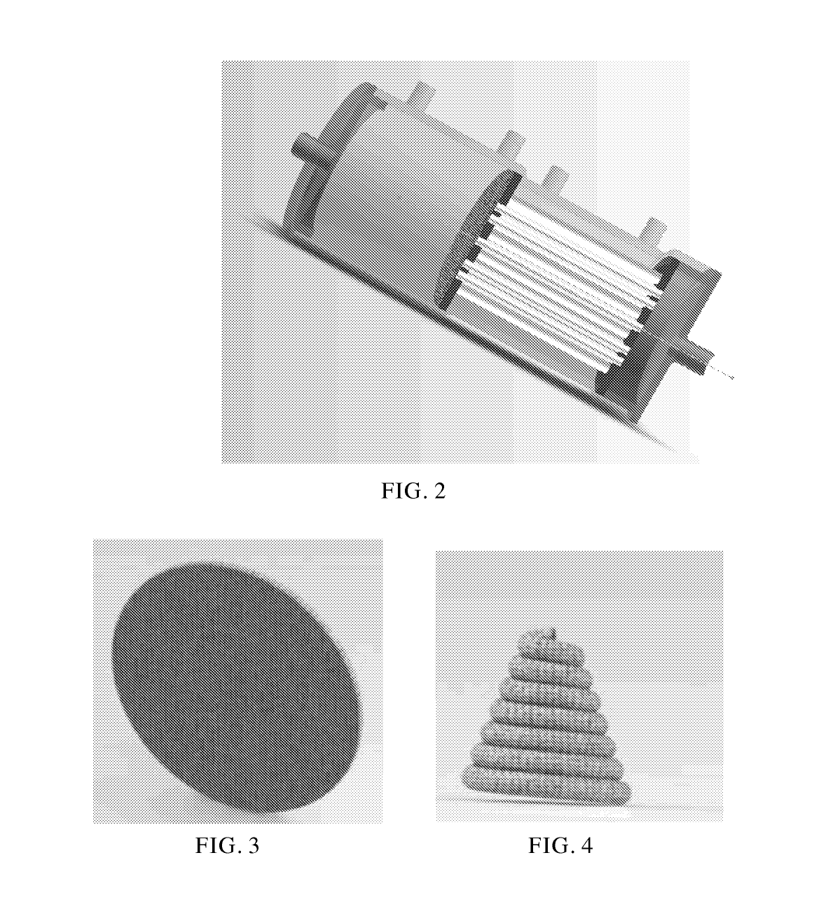

FIG. 2 shows one embodiment of the reaction chamber.

### Example 2

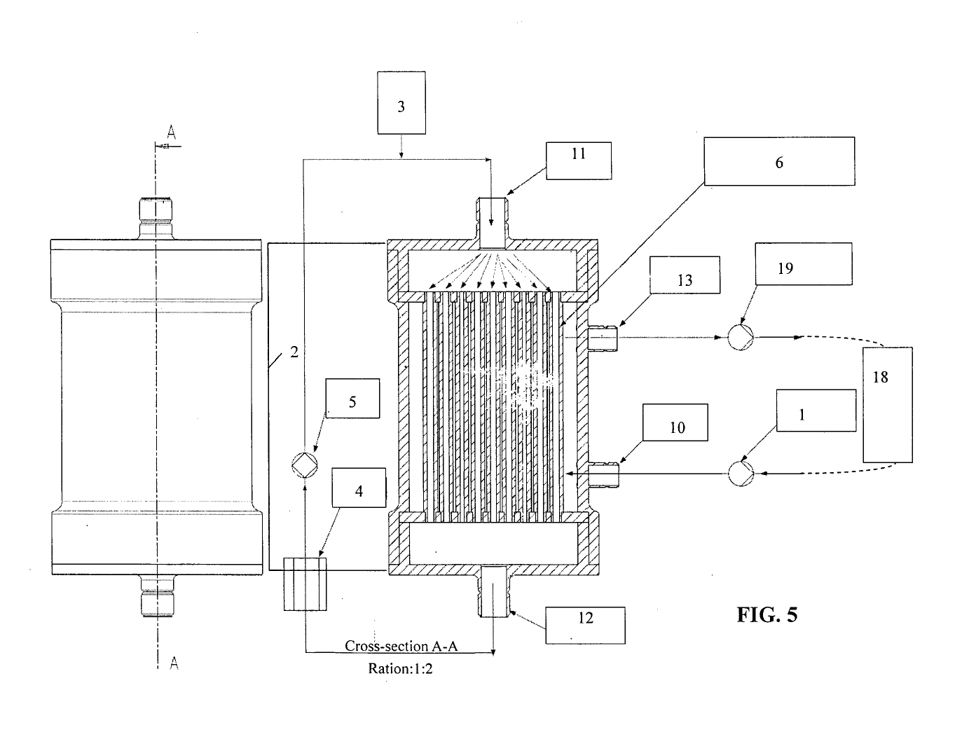

FIG. 5 shows a cross-sectional view of one embodiment of the target-directed, magnetically enhanced system. The system comprises a reaction chamber (2), a pump (1) for pumping untreated blood, a pump (19) for pumping treated blood, a reservoir (3) for supplying non-reacted, magnetic microspheres, a magnet (4), a pump (5) for pumping magnetic microspheres, and a filter element (6). Various components of the system can be connected by catheters or by any means that allow the passage of blood and/or therapeutic solution.

The reaction chamber receives untreated blood from the patient (18) via the first fluid circuit inlet (10), and the treated blood returns back to the patient via the first fluid circuit outlet (13). In one embodiment, a valve is coupled to the first fluid circuit inlet (10) to prevent blood and magnetic microspheres from exiting the reaction chamber through the inlet (10). Pumps (1, 19) are provided to control the flow of blood into, through, and out of the reaction chamber. The reaction chamber also comprises a second fluid circuit inlet (11) for receiving fresh, non-reacted magnetic microspheres, and a second fluid circuit outlet (12) allowing magnetic microspheres to flow out of the reaction chamber. As shown in FIG. 5, patient blood is fed into the reaction chamber in a direction opposite to which magnetic microspheres are fed into the reaction chamber. This counter-current flow facilitates the interaction between blood cells and magnetic microspheres.

A closed second fluid circuit is provided for the circulation of magnetic microspheres into (via inlet (11)), through, and out of (via outlet (12)) the reaction chamber (2). A pump (5) is provided along the second fluid circuit to allow the unidirectional flow of magnetic microspheres. A reservoir (3), which supplies a fresh source of non-reacted magnetic microspheres, is located upstream of the reaction chamber. Preferably, the reservoir is coupled to a valve so that magnetic microspheres can be released into the second fluid circuit at desired time points and in a controlled manner. Before patient blood is fed into the reaction chamber, magnetic microspheres can self-circulate along the second fluid path continuously. After patient blood is fed into the reaction chamber, blood cells can also circulate, with magnetic microspheres, along the second fluid path. In one embodiment, a magnet (4) is positioned along the second fluid circuit. Preferably, a valve is coupled to the magnet. The valve can be opened periodically so that used magnetic microspheres can be removed from the second fluid circuit; meanwhile, fresh, non-reacted magnetic microspheres can be added into the circuit from the reservoir.

A filter element (6) is provided to prevent magnetic microspheres from entering into the patient. The filter element allows the passage of patient blood cells, but completely blocks the passage of magnetic microspheres. In one embodiment as illustrated in FIG. 5, the filter element is comprised of a plurality of filtering tubes. The top lumens of the filtering tubes are in communication with the second fluid circuit inlet (12), through which magnetic microspheres flow into the reaction chamber. The bottom lumens of the filtering tubes are in communication with the second fluid circuit outlet (12), through which magnetic microspheres flow out of the reaction chamber. In this way, magnetic microspheres cannot exit the reaction chamber, via the first fluid circuit inlet (10) and outlet (13), to enter into the patient body.

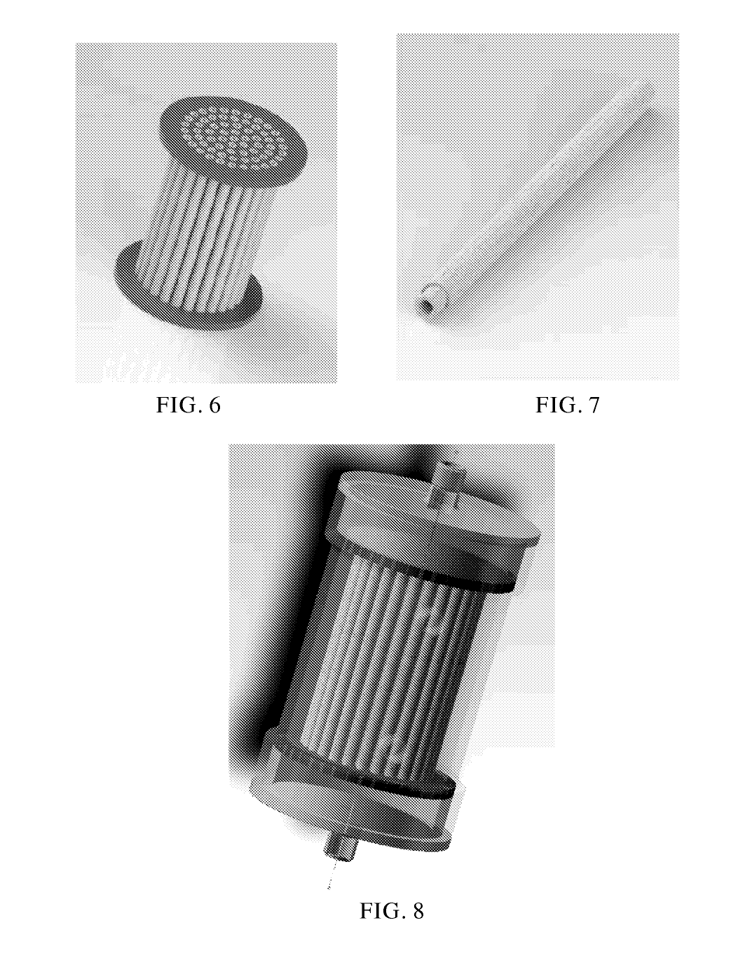

FIG. 6 shows one embodiment of the filter element, comprised of a plurality of filtering tubes. FIG. 7 shows one embodiment of a filtering tube. FIG. 8 shows one embodiment of the reaction chamber.

### Example 3

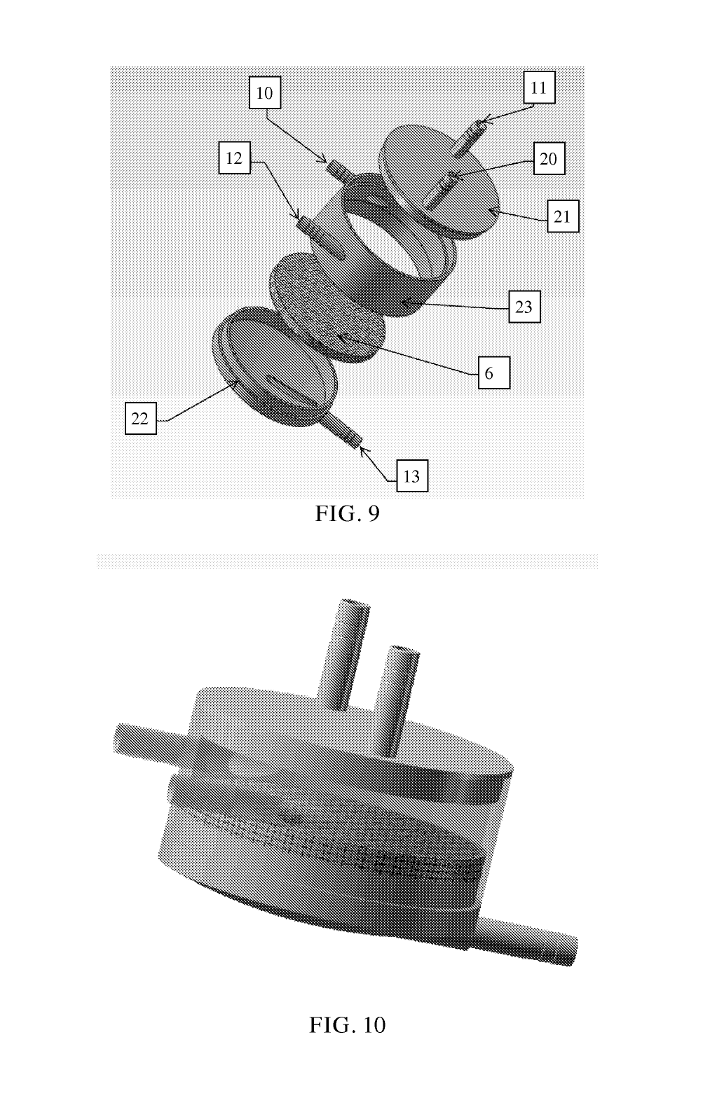

FIG. 9 shows one embodiment of the reaction chamber. The reaction chamber comprises a second fluid circuit inlet (11) for magnetic microspheres, a lumen (20) for inflow of a second therapeutic solution or for emitting substances (such as gas), a first fluid circuit inlet (10) for blood, a second fluid circuit outlet (12) for magnetic microspheres, a first fluid circuit outlet (13) for blood, a top cap (21), a main reaction housing (23), a filter element (6), and a bottom cap (22). In one embodiment, a valve is coupled to the inlet (10) to prevent fluid (including blood) and magnetic microspheres from exiting the reaction chamber through the inlet (10). The filter element (6) is positioned in a sealed manner against the inner walls of the reaction chamber, and separates the reaction chamber into an upper and a lower compartment. In this way, magnetic microspheres are confined in the upper compartment and cannot enter into the patient. Blood cells can pass through the filter element and enter freely into the fluid circuit to react with magnetic microspheres.

During treatment, patient blood enters into the reaction chamber via the blood inlet (10), and the treated blood returns back to the patient via the outlet (13). The magnetic microspheres enter into the reaction chamber via the inlet (11), and exit from the reaction chamber via the outlet (12). To facilitate the interaction between blood cells and magnetic microspheres, the inlet (10) for blood is positioned between the inlet (11) and the outlet (12) for magnetic microspheres, thereby forming a vortex flow of blood-magnetic microspheres. The treated blood passes through the filter element and exits the reaction chamber via the blood outlet (13). In one embodiment, the bottom cap (22) comprises a smooth, convex inner surface and a groove that is connected to the outlet (13). The groove speeds up the return of treated blood back to the patient.

FIG. 10 shows another embodiment of the reaction chamber.

### Example 4

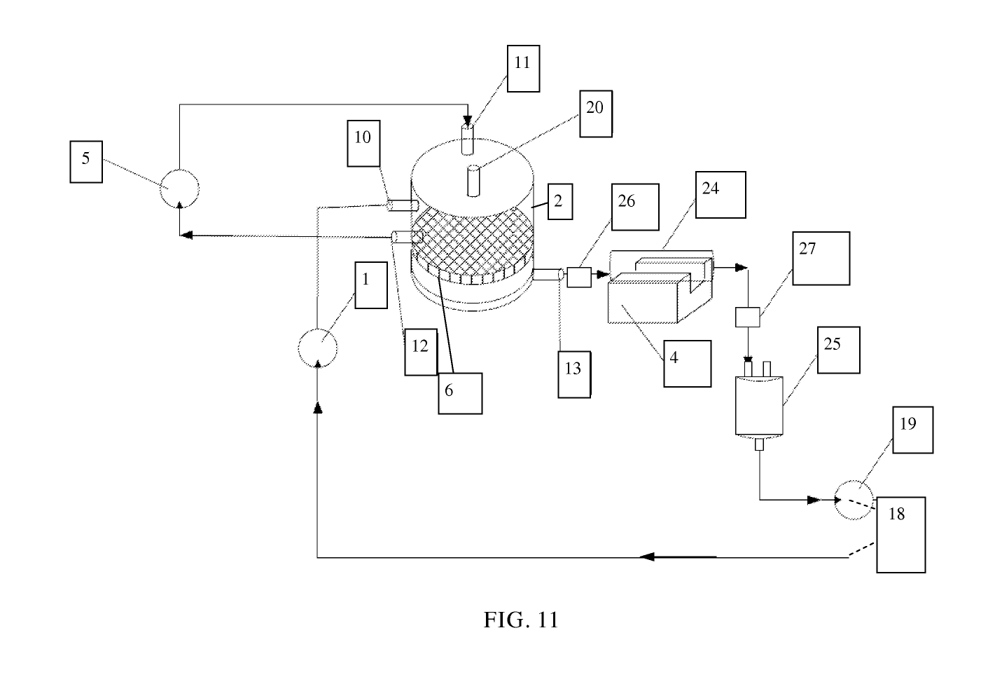

FIG. 11 schematically illustrates one embodiment of the target-specific, magnetically enhanced system of the present invention. The blood treatment device comprises a reaction chamber (2), a first container (24) for storage of treated patient blood, a pump (19) for pumping treated blood back to the patient, a pump (1) for pumping untreated blood to the reaction chamber, a pump (5) for pumping magnetic microspheres, a first monitor (26), a magnet (4), a second container (25) for temporary storage of patient blood, a second monitor (27), and a filter element (6).

The reaction chamber receives untreated blood from the patient via the first fluid circuit inlet (10), and the treated blood is returned back to the patient via the first fluid circuit outlet (13). Pumps (1, 19) are provided to facilitate the flow of blood into, through, and out of the reaction chamber. In one embodiment, a valve is coupled to the inlet (10) to prevent fluid (including blood) and magnetic microspheres from exiting the reaction chamber through the inlet (10). The reaction chamber also comprises a second fluid circuit inlet (11) for receiving fresh, non-reacted magnetic microspheres, a second fluid circuit outlet (12) allowing the magnetic microspheres to flow out of the reaction chamber, and a lumen (20) for receiving a second therapeutic solution or for emitting substances (such as gas). A pump (5) is provided along the fluid circuit to facilitate the unidirectional flow of magnetic microspheres.

A filter element (6) is provided to prevent magnetic microspheres from entering into the patient. As illustrated in FIG. 11, the filter element is positioned in a sealed manner against the inner walls of the reaction chamber, and separates the reaction chamber into an upper and a lower compartment. In this way, magnetic microspheres are confined in the upper compartment and cannot enter into the patient. Blood cells can pass through the filter element and enter freely into the fluid circuit to react with magnetic microspheres.

After the blood exits the reaction chamber, it travels through a first monitor (26), a magnet (4), a second monitor (27), and may be temporarily stored in the second blood container (25) before returning back to the patient. The magnet (4) captures the magnetic microspheres, but does not attract other fluid components, such as patient blood cells. In one embodiment, the magnet (4) is coupled to the first blood container (25). The monitors (26, 27) detect whether the blood contains magnetic microspheres. If the second monitor (27) detects the presence of magnetic microspheres, the blood is returned back to the magnet (4) until all of the magnetic microspheres are completely removed from the blood.

### Example 5

This Example illustrates various embodiments of the device for separation of blood and magnetic microspheres. Blood cells can be separated from magnetic microspheres in various ways. In one embodiment, blood cells are separated from magnetic microspheres using a filter element, which completely blocks the passage of magnetic microspheres, but permits the passage of blood cells across the filter element. In another embodiment, blood cells are separated from magnetic microspheres using a magnet that captures magnetic microspheres, but does not attract other fluid components, such as patient blood cells. In one embodiment, the blood treatment device comprises a plurality of filter elements and/or magnets for separating magnetic microspheres from the blood.

FIGS. 12 and 13 illustrate one embodiment of the separation device. As illustrated in FIG. 13, the separation device comprises a magnetically-based rotating disk (28), catheters (29, 30) coupled to the rotating disk, and a container (31) for storage of magnetic microspheres. In one embodiment, a mixture of blood and magnetic microspheres flows into the magnetically-based rotating disk (28) via the catheter (29). The rotating disk (28) rotates in the same direction as the flow of the fluid. As the fluid mixture passes through the rotating disk, magnetic microspheres adhere to the bottom of the catheter coupled to the rotating disk and enter into the container (31). The magnet does not affect blood flow; as a result, blood passes through the catheter and returns back to the patient via the catheter (30).

FIG. 14 illustrates another embodiment of the separation device, which comprises a magnet (32), a container (33) positioned on top of the magnet, a catheter (34) connected to the lower portion of the container, and a catheter (35) connected to the upper portion of the container. The catheter (34) allows the inflow of fluid mixture containing blood and magnetic microspheres into the container, while the catheter (35) allows the outflow of the blood from the container. The magnet (32) generates a magnetic field that captures magnetic microspheres to the bottom of the container (33). The magnet does not affect blood flow; as a result, blood flows out of the chamber via the catheter (35).

### Example 6

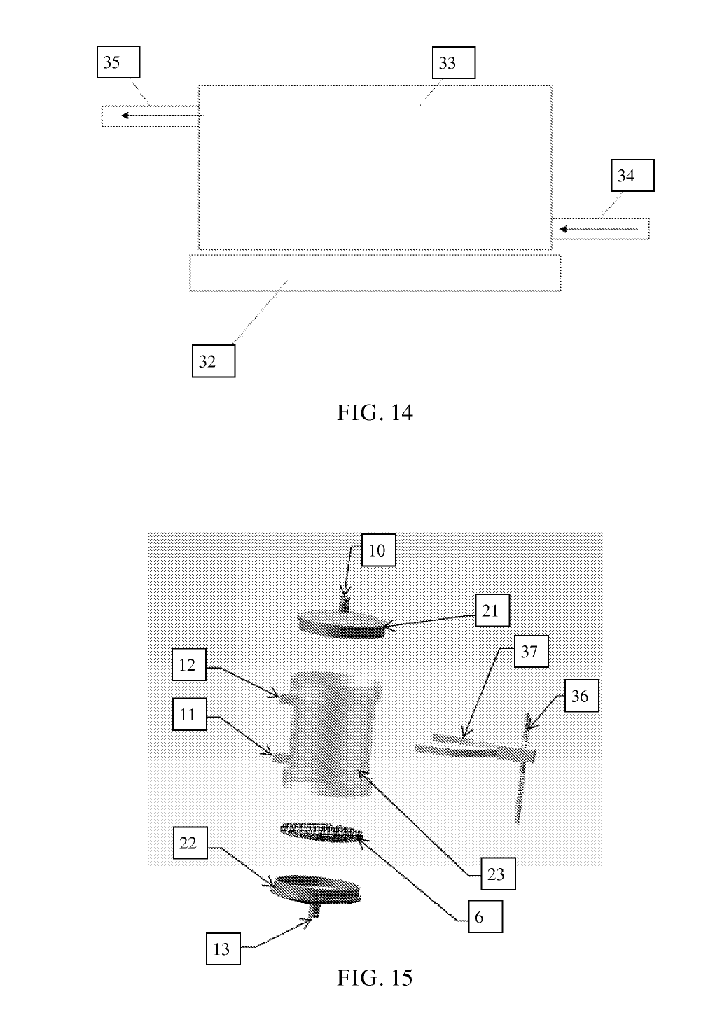

FIG. 15 shows one embodiment of the reaction chamber, which employs an external magnet. The magnet generates a magnetic field so that magnetic microspheres can be stirred continuously during reaction.

As illustrated in FIG. 15, the reaction chamber comprises a first fluid circuit inlet for blood (10), a second fluid circuit inlet (11) for magnetic microspheres, a second fluid circuit outlet (12) for magnetic microspheres, a first fluid circuit outlet (13) for blood, a top cap (21), a main reaction housing (23), a filter element (6), a bottom cap (22), a magnetic coil (36), and a magnetic half-ring (37).

During treatment, patient blood is fed into the reaction chamber via the inlet (10), while the magnetic microspheres circulate in, through, and out of the reaction chamber via the inlet (11) and outlet (12). This allows counter-current flow of blood and magnetic microspheres. In one embodiment, a valve is coupled to the inlet (10) to prevent fluid (including blood) and magnetic microspheres from exiting the reaction chamber through the inlet (10). The blood passes through the filter element (6), and exits from the reaction chamber via the outlet (13).

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

We claim:

1. A target-specific, magnetically enhanced system for removing target molecules from a subject, comprising:
a reaction chamber, comprising:
a first fluid circuit inlet for receiving biological fluid from a subject,
a first fluid circuit outlet for returning the biological fluid back to the subject,
a second fluid circuit outlet allowing the biological fluid to flow out of the reaction chamber and enter into a second fluid circuit, and
a second fluid circuit inlet for returning the biological fluid from the second fluid circuit to the reaction chamber;
a reservoir of magnetic microspheres that bind specifically to a target molecule to be removed from the biological fluid; and
equipment comprising one or more elements that separate the magnetic microspheres from the biological fluid, wherein the equipment allows the passage of the biological fluid but inhibits the passage of the magnetic microspheres, and thereby prevents the magnetic microspheres from entering into the subject;
wherein the system comprises a first fluid circuit for circulation of the biological fluid, wherein the first fluid circuit comprises, in the following order: the first fluid circuit inlet, the reaction chamber, said equipment, and the first fluid circuit outlet; and
wherein the system comprises a second fluid circuit for co-circulation of the biological fluid and the microspheres into, through, and out of the reaction chamber, wherein the second fluid circuit initiates after the first fluid circuit inlet and terminates before the first fluid circuit outlet, wherein the reservoir is positioned along the second fluid circuit.

2. The system, according to claim 1, wherein the biological fluid is blood.

3. The system, according to claim 1, wherein the second fluid circuit terminates before said equipment of the first fluid circuit.

4. The system, according to claim 1, further comprising a magnetic-based device capable of capturing magnetic microspheres, wherein the magnetic-based device is positioned along the second fluid circuit.

5. The system, according to claim 1, further comprising one or more of the following valves:
a) a valve coupled to the first fluid circuit inlet, wherein the valve is positioned to prevent the flow of the biological fluid and/or the magnetic microspheres to the subject;
b) a valve coupled to the first fluid circuit outlet;
c) a valve coupled to the second fluid circuit outlet;
d) a valve coupled to the second fluid circuit inlet;
e) a valve coupled to said equipment of the first fluid circuit; and
f) a valve coupled to the reservoir.

6. The system, according to claim 5, wherein one or more said valves can be controllably opened and closed at a desired time.

7. The system, according to claim 4, further comprising a valve coupled to the magnetic-based device positioned along the second fluid circuit.

8. The system, according to claim 7, wherein said valve can be controllably opened and closed at a desired time.

9. The system, according to claim 1, wherein the element that separates the magnetic microspheres from the biological fluid is selected from a size-based filter or a magnetic-based device capable of capturing magnetic microspheres.

10. The system, according to claim 1, wherein the second fluid circuit further comprises an element that facilitates mixing of the biological fluid and the magnetic microspheres, wherein said element is selected from a magnetic field and/or a stirring element.

11. The system, according to claim 1, further comprising dialysis equipment.

12. The system, according to claim 1, further comprising one or more of the following:
a) a cycler for pumping the biological fluid, wherein said cycler is positioned along the first fluid circuit;
b) a cycler for pumping the magnetic microspheres and/or the biological fluid, wherein said cycler is positioned along the second fluid circuit;
c) a monitor capable of detecting the presence of magnetic microspheres in the biological fluid, wherein the monitor is positioned along the first fluid circuit;
d) equipment for cleaning the system;
e) a waste fluid collector; and
f) a sensor for detecting the presence of magnetic microspheres.

13. The system, according to claim 1, wherein the second fluid circuit inlet is positioned in proximity to the first fluid circuit outlet, and the second fluid circuit outlet is positioned in proximity to the first fluid circuit outlet so that a counter-current flow is formed between the first fluid circuit and the second fluid circuit.

14. A method for removing a target molecule from a subject via extracorporeal circulation of biological fluid of the subject, comprising:
a) receiving biological fluid from a subject, wherein the biological fluid comprises a target molecule to be removed;
b) providing magnetic microspheres that bind specifically to the target molecule to be removed from the biological fluid;
c) directing the biological fluid and the magnetic microspheres to a system comprising:
a reaction chamber, comprising:
a first fluid circuit inlet for receiving the biological fluid from a subject,
a first fluid circuit outlet for returning the biological fluid back to the subject,
a second fluid circuit outlet for directing the biological fluid to flow out of the reaction chamber and enter into a second fluid circuit, and
a second fluid circuit inlet for returning the biological fluid from the second fluid circuit to the reaction chamber;
a reservoir of magnetic microspheres that bind specifically to the target molecule to be removed from the biological fluid; and
equipment comprising one or more elements that separate the magnetic microspheres from the biological fluid, wherein the equipment allows the passage of the biological fluid but inhibits the passage of the magnetic microspheres, and thereby prevents the magnetic microspheres from entering into the subject;
wherein the system comprises a first fluid circuit for circulation of the biological fluid, and the first fluid circuit comprises, in the following order: the first fluid circuit inlet, the reaction chamber, the equipment, and the first fluid circuit outlet; and
wherein the system comprises a second fluid circuit for co-circulation of the biological fluid and the microspheres into, through, and out of the reaction chamber, wherein the second fluid circuit initiates after the first fluid circuit inlet and terminates before the first fluid circuit outlet, wherein the reservoir is positioned along the second fluid circuit; and
d) returning the biological fluid back to the subject.

15. The method, according to claim 14, wherein the biological fluid is blood.

16. The method, according to claim 14, wherein the second fluid circuit ends before said equipment of the first fluid circuit.

17. The method, according to claim 14, wherein the system further comprises a magnetic-based device capable of capturing magnetic microspheres, wherein the magnetic-based device is positioned along the second fluid circuit.

18. The method, according to claim 14, wherein the element that separates the magnetic microspheres from the biological fluid is selected from a size-based filter or a magnetic-based device capable of capturing magnetic microspheres.

19. The method, according to claim 14, wherein the surface of the magnetic microspheres is coated with at least one of the following:
a) an antibody, an antibody fragment, or a fusion protein thereof that bind specifically to the target molecule to be removed from the biological fluid, wherein the target molecule is an antigen;
b) a nucleic acid molecule that hybridizes under stringent conditions to a target molecule, wherein the target molecule is a nucleic acid;
c) a receptor that binds to the target molecule, wherein the target molecule is a ligand; and
d) a ligand that binds to the target molecule, wherein the target molecule is a receptor.

20. The method, according to claim 14, wherein the target molecule is selected from: a) an animal, plant and/or synthetic toxin,
b) an epitope displayed on the surface of a cancer cell,
c) a hormone,
d) a kinase,
e) a pro-inflammatory molecule,
f) a cytokine,
g) an epitope displayed on a viral envelop,
h) a viral nucleic acid molecule,
i) a bacterial nucleic acid molecule, or
j) a bacterial antigen.
